(19) United States
(12) Patent Application Publication
(10) Pub. No.: US 2005/0153976 A1
(43) Pub. Date: Jul. 14, 2005

(54) Pharmaceutical compositions

(75) Inventors: Larry Yun Fang, Plainsboro, NJ (US); Prudence K. Bradley, Cranford, NJ (US); Ping I. Lee, Toronto (CA)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: SCHERING CORPORATION

(21) Appl. No.: 11/013,676

(22) Filed: Dec. 16, 2004

Related U.S. Application Data
(60) Provisional application No. 60/530,466, filed on Dec. 17, 2003.

(51) Int. Cl. A61K 31/496

(52) U.S. Cl. 514/253.11

(57) ABSTRACT

Disclosed herein are novel compositions useful for the treatment of androgen dependant diseases.

### CROSS REFERENCE TO RELATED APPLICATION

This application claims benefit of priority from U.S. Provisional patent application Ser. No. 60/530,466, filed Dec. 17, 2003 which is hereby incorporated by reference in it's entirety.

### BACKGROUND OF THE INVENTION

The invention relates to novel compositions containing hydrophobic inhibitors of Type 3 17β-Hydroxysteroid Dehydrogenase and the use of these compositions for the treatment or prevention of androgen dependent diseases.

Androgen dependent diseases, i.e. diseases whose onset or progress is aided by androgenic activity, are well known. These diseases include but are not limited to prostate cancer, benign prostatic hyperplasia, acne, seborrhea, hirsutism, androgenic alopecia, precocious puberty, adrenal hyperplasia and polycystic ovarian syndrome. Estrogen dependent diseases, i.e. diseases whose onset or progress is aided by estrogenic activity are also well known. These include but are not limited to breast cancer, endometriosis, leiomyoma and precocious puberty.

Androgenic and estrogenic activity may also be reduced by suppressing androgen or estrogen biosynthesis using inhibitors of enzymes that catalyze one or more steps of such biosynthesis. Type 3 17β-Hydroxysteroid Dehydrogenase (17β-HSD3) is the primary enzyme that converts androstenedione to testosterone in the testes. Androgenic and estrogenic activity may also be reduced by suppressing ovarian or testicular secretions by known methods. As such, it would be useful to have agents and compositions containing the same for treating such diseases.

Certain agents useful in treating such disorders must be able to administered to a patient. The aqueous solubility of drug substances plays an important role in the formulation of drug dosage forms. For the oral route of administration it is well experienced that, unless the substance has an aqueous solubility above 10 mg/mL over the pH-range 1-7, then potential absorption problems may occur. A solubility less than 1 mg/mL is likely to give dissolution-rate limited absorption because solubility and dissolution rate are interrelated.

Many important drugs have limited solubility in water, especially hydrophobic drugs. In order to attain the full expected therapeutic effect of such drugs, it is usually required that a solubilized form of the drug be administered to a patient.

A number of methods for solubilizing drugs have been developed that are based on the use of solvents or cosolvents, surfactants, complexation agents (e.g., cyclodextrins, or nicotinamides), or complex drug carriers (e.g., liposomes). Each of the above methods has one or more drawbacks. Conventional surfactants and complexing agents have drawbacks of toxicity, and rapid precipitation of the solubilized drugs once administered to the patient or when otherwise diluted in an aqueous environment. Solvents and cosolvents can be toxic and irritating when injected into humans, such that the use of this solubilization approach is largely restricted to therapies for acute, life threatening diseases where medical experts are constantly in attendance to administer palliative treatments to counteract the adverse effects of the solvents/cosolvents. Water miscible solvents/cosolvents also possess the undesirable feature of allowing the drug to rapidly precipitate when an aqueous environment is contacted. Complex drug carriers, such as liposomes have limited utility due to the unstable nature of the carrier particles and the preferential uptake and localization of liposomal drugs to the reticuloendothelial system, namely, the liver and spleen. Accordingly, there exists a need for compositions that do not suffer from the above mentioned infirmities that can deliver a hydrophobic compound, such as the compound of Formula I below, that are useful for the treatment of androgen related diseases and compositions that have improved stability over time.

### SUMMARY OF THE INVENTION

Accordingly, there is disclosed a pharmaceutically acceptable composition comprising an effective amount of the compound represented by the chemical structural formula I comprising: 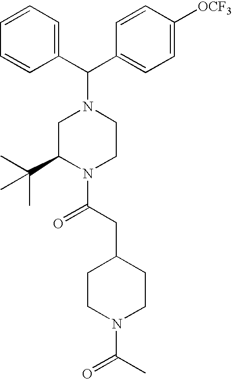
in admixture with a hydrophilic carrier selected from the group consisting of silica and microcrystalline cellulose, wherein said compound represented by the chemical structural formula I is adsorbed onto said hydrophilic carrier.

There is also disclosed a pharmaceutically acceptable composition comprising an effective amount of the compound represented by the chemical structural formula 11 comprising
Formula (II): 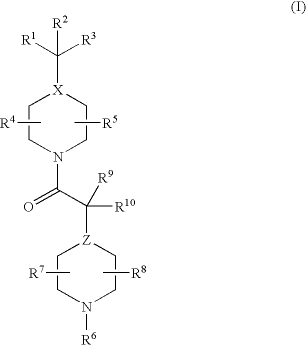
a prodrug thereof, or a pharmaceutically acceptable salt or solvate of the compound or of said prodrug wherein,
R¹ and R² are the same or different and are independently selected from the group consisting of aryl, heteroaryl, arylalkyl, and heteroarylalkyl, each optionally substituted with one to six groups selected from the group consisting of:
a) halogen;
b) —OCF₃ or —OCHF₂
c) —CF₃;
d) —CN;
e) alkyl or R¹⁸-alkyl;
f) heteroalkyl or R¹⁸-heteroalkyl;
g) aryl or R¹⁸-aryl;
h) heteroaryl or R¹⁸-heteroaryl;
i) arylalkyl or R¹⁸-arylalkyl;
j) heteroarylalkyl or R¹⁸-heteroarylalkyl
k) hydroxy;
l) alkoxy;
m) aryloxy;
n) —SO₂-alkyl;
o)—NR¹¹R¹²;
p) —N(R¹¹)C(O)R¹³,
q) methylenedioxy;
r) difluoromethylenedioxy;
s) trifluoroalkoxy;
t) —SCH₃ or —SCF₃; and
u) —SO₂CF₃ or —NHSO₂CF₃;
R³ is H, —OH, alkoxy or alkyl, provided that when X is N, R³ is not —OH or alkoxy;
R⁴, R⁵, R⁷ and R⁸ are the same or different and are independently selected from the group consisting of: H, —OH, —OR¹⁴, —NR¹¹R¹², —N(R¹¹)C(O)R¹³, alkyl, aryl, cycloalkyl, arylalkyl, heteroalkyl, heteroaryl, heteroarylalkyl, heterocycloalkyl, 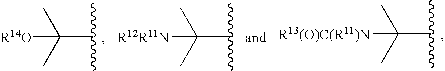
provided that when Z and/or X is N, then R⁴, R⁵, R⁷ and R⁸ are each not —OH, —OR¹⁴, —NR¹¹R¹² or —N(R¹¹)C(O)R¹³;
R⁶ is selected from the group consisting of —C(O)R¹⁵ and —SO₂R¹⁵;
R⁹ and R¹⁰ are the same or different and are independently selected from the group consisting of: H, F, —CF₃, alkyl, cycloalkyl, arylalkyl, heteroalkyl, heteroarylalkyl, heterocycloalkyl, hydroxy, alkoxy, aryloxy, —NR¹¹_{R}¹² and —N(R¹¹)C(O)R¹³; provided that when Z is N, then R⁹ and R¹⁰ are each not F, hydroxy, alkoxy, aryloxy, —NR¹¹R¹² or —N(R¹¹)C(O)R¹³;
R¹¹ is selected from the group consisting of H, alkyl, aryl and heteroaryl;
R¹² is selected from the group consisting of H, alkyl, aryl and heteroaryl;
R¹³ is selected from the group consisting of alkyl, alkoxy and aryloxy;
R¹⁴ is selected from the group consisting of H, alkyl, aryl and heteroaryl;
R¹⁵ is selected from the group consisting of: —NR¹⁶R¹⁷, —OR¹⁶, alkyl, cycloalkyl, heterocycloalkyl, aryl, arylalkyl and heteroarylalkyl, each optionally substituted with R¹⁸;
R¹⁶ and R¹⁷ are the same or different and are independently selected from the group consisting of: H, alkyl, aryl, arylalkyl, heteroalkyl and heteroaryl, each optionally substituted with R¹⁸, provided that when R¹⁵ is —OR¹⁶, R¹⁶ is not H;
R¹⁸ is one to four substituents each independently selected from the group consisting of: lower alkyl, halo, cyano, nitro, haloalkyl, hydroxy, alkoxy, alkoxy carbonyl, carboxy, carboxyalkyl, carboxamide, mercapto, amino, alkylamino, dialkylamino, sulfonyl, sulfonamido, cycloalkyl, heterocycloalkyl, heterocycloalkylalkyl, aryl and heteroaryl; and
X and Z are the same or different and are independently selected from the group consisting of C and N or a pharmaceutically acceptable salt thereof, in admixture with a hydrophilic carrier selected from the group consisting of silica and microcrystalline cellulose, wherein said compound represented by the chemical structural formula I is adsorbed onto said hydrophilic carrier.

### DETAILED DESCRIPTION OF THE INVENTION

Accordingly, the present invention is directed to a pharmaceutically acceptable composition comprising an effective amount of the compound represented by the chemical structural formula I comprising: 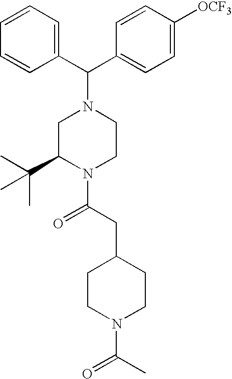
in admixture with a hydrophilic carrier selected from the group consisting of silica and microcrystalline cellulose, wherein said compound represented by the chemical structural formula I is adsorbed onto said hydrophilic carrier.

A particularly useful compound for use in the present invention is the compound represented by the chemical structural formula I comprising: 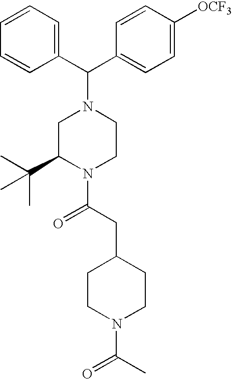
This compound is chemically known as 1-[(1-acetyl-4-piperidinyl)acetyl]-2(S)-(1,1-dimethylethyl)-4(S)*4-[phenyl [4(trifluoromethoxy)phenyl]methyl]piperazine and has a molecular weight of 559.3 g/mole. It is a weak base with a pKa of 4.0 corresponding to the ionization of the piperazine ring. The value of log P is 3.0 and the log D at pH 7.4 is calculated to be 2.9. The solubility of this compound is less than 1 μg/mL (2 μM) at a pH greater than 7.4. The solubility increases to 1.9 μg/mL (3.4 μM) at pH 4.0 and to 74.6 μg/mL (133 μM) at pH 2.3. The compound is very soluble in both ethanol and methanol (>150 mg/mL). The solubility in most common organic solvents, such as acetonitrile, dimethyl sulfoxide (DMSO), methylene chloride, ethyl acetate, isopropyl alcohol, hexane, and acetone, generally exceeds 50 mg/mL. The free form of the compound is amorphous, but a crystalline hydrogen sulfate salt of this compound can be made. The X-ray powder diffraction (XRPD) patterns of the amorphous free base and the crystalline hydrogen sulfate salt are shown in FIG. 1.

The compound is useful in the treatment of androgen dependent diseases. It may be prepared in accordance with the procedures set forth in U.S. patent application Ser. No. 10/235,627, filed Sep. 5, 2002, and Ser. No. 10/271,358, filed Oct. 15, 2002, which are incorporated herein by reference in their entirety. The compound may be present in the compositions of the present invention in amounts of about 20% to about 75%, preferably about 34% to about 50%.

In another aspect of the present invention, there is disclosed compounds represented by Formula (II): 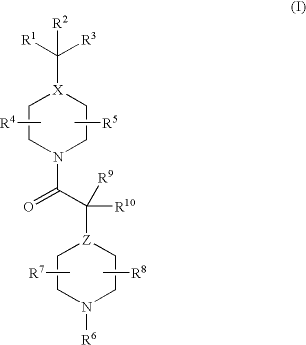
a prodrug thereof, or a pharmaceutically acceptable salt or solvate of the compound or of said prodrug wherein,
R¹ and R² are the same or different and are independently selected from the group consisting of aryl, heteroaryl, arylalkyl, and heteroarylalkyl, each optionally substituted with one to six groups selected from the group consisting of:
a) halogen;
b) —OCF₃ or —OCHF₂
c) —CF₃;
d) —CN;
e) alkyl or R¹⁸-alkyl;
f) heteroalkyl or R¹⁸-heteroalkyl;
g) aryl or R¹⁸-aryl;
h) heteroaryl or R¹⁸-heteroaryl;
i) arylalkyl or R¹⁸-arylalkyl;
j) heteroarylalkyl or R¹⁸-heteroarylalkyl;
k) hydroxy;
l) alkoxy;
m) aryloxy;
n) —SO₂-alkyl;
o) —NR¹¹R¹²;
p) —N(R¹¹)C(O)R¹³,
q) methylenedioxy;
r) difluoromethylenedioxy;
s) trifluoroalkoxy;
v) —SCH₃ or —SCF₃; and
w) —SO₂CF₃ or —NHSO₂CF₃;
R³ is H, —OH, alkoxy or alkyl, provided that when X is N, R³ is not —OH or alkoxy;
R⁴, R⁵, R⁷ and R⁸ are the same or different and are independently selected from the group consisting of: H, —OH, —OR¹⁴, —NR¹¹R¹², —N(R¹¹)C(O)R¹³, alkyl, aryl, cycloalkyl, arylalkyl, heteroalkyl, heteroaryl, heteroarylalkyl, heterocycloalkyl, 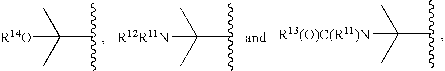
provided that when Z and/or X is N, then R⁴, R⁵, R⁷ and R⁸ are each not —OH, —OR¹⁴, —NR¹¹R¹² or —N(R¹¹)C(O)R¹³;
R⁶ is selected from the group consisting of —C(O)R¹⁵ and —SO₂R¹⁵;
R⁹ and R¹⁰ are the same or different and are independently selected from the group consisting of: H, F, —CF₃, alkyl, cycloalkyl, arylalkyl, heteroalkyl, heteroarylalkyl, heterocycloalkyl, hydroxy, alkoxy, aryloxy, —NR¹¹R¹² and —N(R¹¹)C(O)R¹³; provided that when Z is N, then R⁹ and R¹⁰ are each not F, hydroxy, alkoxy, aryloxy, —NR¹¹R¹² or —N(R¹¹)C(O)R¹³;
R¹¹ is selected from the group consisting of H, alkyl, aryl and heteroaryl;
R¹² is selected from the group consisting of H, alkyl, aryl and heteroaryl;
R¹³ is selected from the group consisting of alkyl, alkoxy and aryloxy;
R¹⁴ is selected from the group consisting of H, alkyl, aryl and heteroaryl;
R¹⁵ is selected from the group consisting of: —NR¹⁶R¹⁷, —OR¹⁶, alkyl, cycloalkyl, heterocycloalkyl, aryl, arylalkyl and heteroarylalkyl, each optionally substituted with R¹⁸;
R¹⁶ and R¹⁷ are the same or different and are independently selected from the group consisting of: H, alkyl, aryl, arylalkyl, heteroalkyl and heteroaryl, each optionally substituted with R¹⁸, provided that when R¹⁵ is —OR¹⁶, R¹⁶ is not H;
R¹⁸ is one to four substituents each independently selected from the group consisting of: lower alkyl, halo, cyano, nitro, haloalkyl, hydroxy, alkoxy, alkoxy carbonyl, carboxy, carboxyalkyl, carboxamide, mercapto, amino, alkylamino, dialkylamino, sulfonyl, sulfonamido, cycloalkyl, heterocycloalkyl, heterocycloalkylalkyl, aryl and heteroaryl; and
X and Z are the same or different and are independently selected from the group consisting of C and N, in admixture with a hydrophilic carrier selected from the group consisting of silica and microcrystalline cellulose, wherein said compound represented by the chemical structural formula I is adsorbed onto said hydrophilic carrier.

This formulation delivery system utilizes hydrophilic carriers, such as SiO₂ or microcrystalline cellulose (MCC), as carriers to adsorb hydrophobic or lipophilic compounds (such as the compound of structural formula I) onto its surface and to suspend the composition in aqueous medium.

Fine-particle size silica or SiO₂ is a commercially available and useful pharmaceutically inert material. Among commercially available fine-particle silicas are those, e.g., bearing the tradenames, such as, for example, Syliod® 244 available from W. R. Grace & Co. The SiO₂ may be present in the compositions of the present invention in amounts of about 10% to about 80%, preferably about 40% to about 60%,

Another suitable carrier includes microcrystalline cellulose (MCC) such as Avicel® PH102, Avicel® PH105, all available from FMC Corporation. The MCC may be present in the compositions of the present invention in amounts of about 10% to about 90%, preferably about 60% to about 75%.

Suitable non-limiting aqueous solutions for use in the present invention include, for example, 0.4% hydroxypropyl methylcellulose (HPMC) solution and water.

SiO₂ and MCC have characteristic physicochemical properties, such as high ratio values of surface area to mass, very large surface areas, excellent flow properties, fine particle size and a hydrophilic surface capable of hydrogen bonding which enhances suspension in aqueous media. Hydrophobic or lipophilic molecules, including the compound of the above structural formula, are usually very difficult to suspend in aqueous solutions. In the present case, the compound of the above structural formula forms a gum when suspended in aqueous media.

Hydrophobic or lipophilic compounds may be adsorbed on to inert surfaces by deposition and precipitation. The first step is to dissolve the compound in a volatile solvent, such as ethanol or methanol, followed by blending with inert material such as SiO₂ or MCC. Drying is completed by heating the mixture gradually, for example, from 25° C. to 50° C. over 1 hour and maintaining at 50° C. overnight under house vacuum.

With proper ratios of carrier to compound, free flowing and uniform powders or solids can be produced. These powders or solids should have the original molecular characteristics of the adsorbed chemicals. The residual solvent (EtOH) used in this process can be reduced to 20 ppm level. These flowable powders or solids are suspended in an aqueous solution, such as 0.4% HPMC solution or water.

The compositions of the present invention may contain the compound or active pharmaceutical ingredient (API) and carrier in a ratio of about 1:1 to about 1:5 for SiO₂ and about 1:1.5 to about 1:5 for MCC. The lowest ratio of compound to carrier which produces flowable powder suitable for producing stable suspensions in aqueous mediums is 1:1 for SiO₂ and 1:1.5 for MCC. The preferred ratio should be 1:2 for both SiO₂ and MCC.

The flowable powders or solids have been tested to be very stable for at least two weeks chemically and physically at room temperature (RT) and ICH RH4 conditions (40° C. and 75% relative humility). The suspension solutions (SiO₂-Compound or MCC-Compound in 0.4% HPMC solution or water) are also very stable both chemically and physically for at least two weeks at room temperature. The evaluation of SiO₂-Compound and MCC-Compound are shown in Example 1.

### EXAMPLE 1

| Evaluation | SiO₂-Compound | | MCC-Compound | |
|---|---|---|---|---|
| Ratio of | 2:1 | 1:1 | 2:1 | 1.5:1 |
| Carrier:API | | | | |
| Solid State | Flowable | Flowable | Flowable | Flowable |
| | Powder | Powder | Powder | Powder/Solid |
| EtOH Residual | <20 ppm | <20 ppm | <20 ppm | <20 ppm |
| Solid Powder | >2-weeks | >2-weeks | >2-weeks | >2-weeks |
| Chemical | (RT, RH4) | (RT, RH4) | (RT, RH4) | (RT, RH4) |
| Stability | | | | |
| pH in 0.4% | 6.0 | 6.1 | 6.1 | 6.1 |
| HPMC Solution | | | | |
| Suspension | Stable | Stable | Stable | Stable |
| Chemical | >2-weeks | >2-weeks | >2-weeks | >2-weeks |
| Stability | | | | |
| Suspension | Excellent | Excellent | Excellent | Excellent |
| Physical Stability | >4-weeks | >4-weeks | >4-weeks | >4-weeks |
| Re-suspensibility | Excellent | Excellent | Excellent | Excellent |
| | >6-weeks | >6-weeks | >6-weeks | >6-weeks |

In-vitro dissolution testing in 0.1 N HCl of this composition demonstrate that the compound can be released quickly and higher than 90% recovery has been observed within one and a half hour in 0.1 N HCl solution.

In-vivo pharmacokinetic (PK) studies in rats and dogs with these compositions show better PK results in terms of AUC, Cmax, and dose proportionality than that of compound only. The results are set forth in Table 2.

### EXAMPLE 2

| Dose | | | |
|---|---|---|---|
| formulation | SiO₂-API | MCC-API | API |
| Carrier:API | 1:1 | 1.5:1 | 0:1 |
| Dose (mg/kg) | 10 | 10 | 10 |
| AUC (0-t*) | 4090 | 3060 | 2520 |
| (ng · hr/mL) | | | |
| AUC (infinity) | 4170 | 3130 | 2550 |
| (ng · hr/mL) | | | |
| AUC (0-t*) | 7.3 | 5.5 | 4.5 |
| (μM · hr) | | | |
| AUC (infinity) | 7.4 | 5.6 | 4.6 |
| (μM · hr) | | | |
| % AUC (infinity) | 1.9 | 2.2 | 1.2 |
| Cmax (ng/mL) | 410 | 330 | 260 |
| Cmax (μM) | 0.7 | 0.6 | 0.5 |
| Tmax (hr) | 3.3 | 2.0 | 3.3 |

The compositions of the present invention offer a number of significant advantages. The compositions of the present invention form a solid flowable powder to be formed by adsorbing hydrophobic or lipophilic compounds onto hydrophilic materials having a very high surface area, such as SiO₂ and MCC, as drug delivery material, as well as the processes for preparing the same. More specifically, the compositions of the present inventions also form solid flowable powder compositions for new chemical compounds, such as the compound represented by the chemical structural formula I formed with these carriers.

The term “pharmaceutically-acceptable excipients,” as used herein, includes any physiologically inert, pharmacologically inactive material known to one skilled in the art, which is compatible with the physical and chemical characteristics of the particular active ingredient selected for use. Pharmaceutically-acceptable excipients include polymers, resins, plasticizers, fillers, binders, lubricants, glidants, disintegrates, solvents, co-solvents, buffer systems, surfactants, preservatives, sweetening agents, flavoring agents, pharmaceutical grade dyes or pigments, and viscosity agents.

The present invention will be further described with reference to the following non-limiting examples.

### EXAMPLE 3

Preparation of SiO₂-Compound of Formula I samples may be carried out by the following representative steps. Uniformly mix 0.5 grams of API free form (amorphous) with 1.0 grams of SiO₂ (Syliod®) 244 FP) in a container using a magnetic stirrer. Then add 5.0 mL of ethanol to the container to dissolve the compound completely. Stir at room temperature and dry the solution under N₂ gas until a slurry is formed. Dry the slurry at increasing temperature beginning at 25° C. ending at 50° C. over a duration of 1 hour and at 50° C. for overnight under house vacuum. The slurry is dried to a flowable powder containing adsorbed compound. The amount of ethanol was less than 20 ppm in the sample using GC-MS determination. The sample was tested to be chemically stable at RT and RH4 condition for at least four weeks. It was well suspended in 0.4% HPMC solution at 100 mg/mL and the suspension stayed physically and chemically stable for at least four weeks.

### EXAMPLE 4

Preparation of MCC-Compound of Formula I samples may be carried out by the following representative steps. Uniformly mix 0.5 grams of compound free form (amorphous) with 1.0 grams of MCC (Avicel® PH105) in a container using a magnetic stirrer. The remainder of the procedure is the same as that described in the preparation procedure of Example 1 above. The amount of ethanol was less than 20 ppm in the sample based on GC-MS determination. The sample was tested to be chemically stable at RT and RH4 condition for at least four weeks. The sample was well suspended in 0.4% HPMC solution at 102 mg/mL. The suspension stayed physically and chemically stable for at least four weeks. A reference sample of compound in the free form without being adsorbed onto the carrier was also suspended in 0.4% HPMC solution for comparison. Since the compound in free form is weakly basic and lipophilic, the compound without being adsorbed onto the carrier forms a gum and is adsorbed onto all contacting surfaces of the container.

Many modifications and variations of this invention can be made without departing from its spirit and scope, as will be apparent to one skilled in the art. The specific embodiments described herein are offered by way of example only, and the invention is to be limited only by the terms of the appended claims along with the full scope of equivalents to which such claims are entitled.

1. A pharmaceutically acceptable composition comprising an effective amount of the compound represented by the chemical structural formula I comprising: 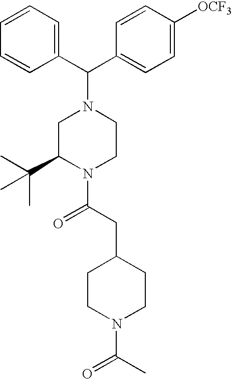
or a pharmaceutically acceptable salt thereof, in admixture with a hydrophilic carrier selected from the group consisting of silica and microcrystalline cellulose, wherein said compound represented by the chemical structural formula I is adsorbed onto said hydrophilic carrier.

2. The pharmaceutically acceptable composition according to claim 1, wherein the compound represented by the chemical structural formula I is present in an amount of about 20% to about 75%.

3. The pharmaceutically acceptable composition according to claim 1, wherein the compound represented by the chemical structural formula I is present in an amount of about 34% to about 50%.

4. The pharmaceutically acceptable composition according to claim 1, wherein the hydrophilic carrier is present in an amount of about 10% to about 90%.

5. The pharmaceutically acceptable composition according to claim 1, wherein the hydrophilic carrier is microcrystalline cellulose.

6. The pharmaceutically acceptable composition according to claim 5, wherein the microcrystalline cellulose is present in an amount of about 10% to about 90%.

7. The pharmaceutically acceptable composition according to claim 6, wherein the microcrystalline cellulose is present in an amount of about 60% to about 75%.

8. The pharmaceutically acceptable composition according to claim 1, wherein the hydrophilic carrier is silica.

9. The pharmaceutically acceptable composition according to claim 6, wherein the silica is present in an amount of about 10% to about 80%.

10. The pharmaceutically acceptable composition according to claim 9, wherein the silica is present in an amount of about 40% to about 60%.

11. The pharmaceutically acceptable composition according to claim 1, wherein the ratio compound represented by the chemical structural formula I to hydrophilic carrier is about 1:1 to about 1:5.

12. The pharmaceutically acceptable composition according to claim 11, wherein the ratio compound represented by the chemical structural formula I to hydrophilic carrier is about 1:1.

13. The pharmaceutically acceptable composition according to claim 11, wherein the ratio compound represented by the chemical structural formula I to hydrophilic carrier is about 1:1.5.

14. The pharmaceutically acceptable composition according to claim 1, wherein the ratio compound represented by the chemical structural formula I to hydrophilic carrier is about 1:2.

15. The pharmaceutically acceptable composition according to claim 1 further comprising an aqueous solvent.

16. The pharmaceutically acceptable composition according to claim 15, wherein the aqueous solvent is 0.4% HPMC solution or water.

17. The pharmaceutically acceptable composition according to claim 1 further comprising a pharmaceutically acceptable excipient.

18. The pharmaceutically acceptable composition according to claim 17, wherein the pharmaceutically-acceptable excipients comprise polymers, resins, plasticizers, fillers, binders, lubricants, glidants, disintegrates, solvents, co-solvents, buffer systems, surfactants, preservatives, sweetening agents, flavoring agents, pharmaceutical grade dyes or pigments, and viscosity agents.

19. A pharmaceutically acceptable composition comprising an effective amount of the compound represented by the chemical structural Formula II comprising
Formula (II): 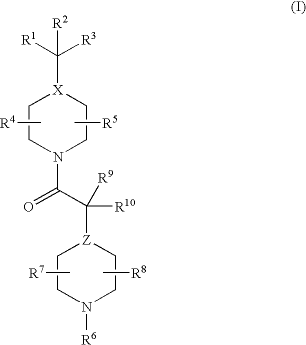
a prodrug thereof, or a pharmaceutically acceptable salt or solvate of the compound or of said prodrug wherein,
R¹ and R² are the same or different and are independently selected from the group consisting of aryl, heteroaryl, arylalkyl, and heteroarylalkyl, each optionally substituted with one to six groups selected from the group consisting of:
a) halogen;
b) —OCF₃ or —OCHF₂
c) —CF₃;
d) —CN;
e) alkyl or R¹⁸-alkyl;
f) heteroalkyl or R¹⁸-heteroalkyl;
g) aryl or R¹⁸-aryl;
h) heteroaryl or R¹⁸-heteroaryl;
i) arylalkyl or R¹⁸-arylalkyl;
j) heteroarylalkyl or R¹⁸-heteroarylalkyl
k) hydroxy;
l) alkoxy;
m) aryloxy;
n) —SO₂-alkyl;
o) —NR¹¹R¹²;
p) —N(R¹¹)C(O)R¹³,
q) methylenedioxy;
r) difluoromethylenedioxy;
s) trifluoroalkoxy;
x) —SCH₃ or —SCF₃; and
y) —SO₂CF₃ or —NHSO₂CF₃;
R³ is H, —OH, alkoxy or alkyl, provided that when X is N, R³ is not —OH or alkoxy;
R⁴, R⁵, R⁷ and R⁸ are the same or different and are independently selected from the group consisting of: H, —OH, —OR⁴, —NR¹¹R¹², —N(R¹¹)C(O)R¹³, alkyl, aryl, cycloalkyl, arylalkyl, heteroalkyl, heteroaryl, heteroarylalkyl, heterocycloalkyl, 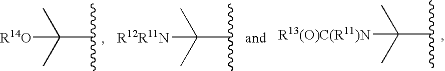
provided that when Z and/or X is N, then R⁴, R⁵, R⁷ and R⁸ are each not —OH, —OR¹⁴, —NR¹¹R¹² or —N(R¹¹)C(O)R¹³;
R⁶ is selected from the group consisting of —C(O)R¹⁵ and —SO₂R¹⁵;
R⁹ and R¹⁰ are the same or different and are independently selected from the group consisting of: H, F, —CF₃, alkyl, cycloalkyl, arylalkyl, heteroalkyl, heteroarylalkyl, heterocycloalkyl, hydroxy, alkoxy, aryloxy, —NR¹¹R¹² and —N(R¹¹)C(O)R¹³; provided that when Z is N, then R⁹ and R¹⁰ are each not F, hydroxy, alkoxy, aryloxy, —NR¹¹R¹² or —N(R¹¹)C(O)R¹³;
R¹¹ is selected from the group consisting of H, alkyl, aryl and heteroaryl;
R¹² is selected from the group consisting of H, alkyl, aryl and heteroaryl;
R¹³ is selected from the group consisting of alkyl, alkoxy and aryloxy;
R¹⁴ is selected from the group consisting of H, alkyl, aryl and heteroaryl;
R¹⁵ is selected from the group consisting of: —NR¹⁶R¹⁷, —OR¹⁶, alkyl, cycloalkyl, heterocycloalkyl, aryl, arylalkyl and heteroarylalkyl, each optionally substituted with R¹⁸;
R¹⁶ and R¹⁷ are the same or different and are independently selected from the group consisting of: H, alkyl, aryl, arylalkyl, heteroalkyl and heteroaryl, each optionally substituted with R¹⁸, provided that when R¹⁵ is —OR¹⁶, R¹⁶ is not H;
R¹⁸ is one to four substituents each independently selected from the group consisting of: lower alkyl, halo, cyano, nitro, haloalkyl, hydroxy, alkoxy, alkoxy carbonyl, carboxy, carboxyalkyl, carboxamide, mercapto, amino, alkylamino, dialkylamino, sulfonyl, sulfonamido, cycloalkyl, heterocycloalkyl, heterocycloalkylalkyl, aryl and heteroaryl; and
X and Z are the same or different and are independently selected from the group consisting of C and N or a pharmaceutically acceptable salt thereof, in admixture with a hydrophilic carrier selected from the group consisting of silica and microcrystalline cellulose, wherein said compound represented by the chemical structural formula I is adsorbed onto said hydrophilic carrier.

20. The pharmaceutically acceptable composition according to claim 19, further comprising a pharmaceutically acceptable excipient.
